(19) United States
(12) Patent Application Publication
(10) Pub. No.: US 2010/0098656 A1
(43) Pub. Date: Apr. 22, 2010

(54) Polyamino acids functionalized by hydrophobic grafts bearing an anionic charge and applications thereof, such as therapeutic applications

(75) Inventors: Olivier Breyne, Lyon (FR); Gauthier Pouliquen, Lyon (FR); You-Ping Chan, Lyon (FR)

Correspondence Address: PATTON BOGGS LLP, 8484 WESTPARK DRIVE, SUITE 900, MCLEAN, VA, 22102, US

(73) Assignee: Flamel Technologies, Venissieux (FR)

(21) Appl. No.: 11/658,803

(22) PCT Filed: Jul. 25, 2005
(86) PCT No.: PCT/FR2005/050610

§ 371 Date: Nov. 17, 2009

(30) Foreign Application Priority Data
Jul. 30, 2004 (FR) 0408478

(51) Int. Cl. A61K 38/16; C07K 2/00; A61K 38/18; A61K 38/20; A61K 38/21

(52) U.S. Cl. 424/85.2; 530/300; 514/12; 424/85.4

(57) ABSTRACT

The present invention relates to novel materials based on biodegradable polyamino acids that are useful especially for the vectorization of active principle(s) (AP). The invention further relates to novel pharmaceutical, cosmetic, dietetic or phytosanitary compositions based on these polyamino acids. The object of the invention is to provide a novel polymer starting material that is capable of being used for the vectorization of AP and makes it possible on the one hand to achieve high polymer/AP ratios, and on the other hand optimally to satisfy all the specifications required in the case in point: biocompatibility, biodegradability, ability to associate easily with numerous active principles or to solubilize them, and ability to release these active principles in vivo. This object is achieved by the present invention, which relates first and foremost to linear polyamino acids comprising aspartic units or glutamic units and having hydrophobic grafts comprising hydrophobic groups containing from 8 to 30 carbon atoms, at least one of these hydrophobic grafts having at least one anionic charge and/or one or more mutually identical or different ionizable groups each capable of giving rise to at least one anionic charge. These polymers are amphiphilic and anionic and are capable of being converted easily and economically to particles for the vectorization of active principles, these particles themselves being capable of forming stable aqueous colloidal suspensions.

The present invention relates to novel materials based on biodegradable polyamino acids that are useful especially for the vectorization of one or more active principles (AP).

The invention further relates to novel pharmaceutical, cosmetic, dietetic or phytosanitary compositions based on these polyamino acids. These compositions can be of the type allowing the vectorization of AP and preferably taking the form of emulsions, micelles, particles, gels, implants or films.

The AP in question are advantageously biologically active compounds which can be administered to an animal or human organism by the oral, parenteral, nasal, vaginal, ocular, subcutaneous, intravenous, intramuscular, intradermal, intraperitoneal, intracerebral, buccal or other route.

The AP to which the invention relates more particularly, but without implying a limitation, are proteins, glycoproteins, peptides, polysaccharides, lipopolysaccharides, oligonucleotides or polynucleotides, and organic molecules. However, they can also be cosmetic products or phytosanitary products such as herbicides, insecticides, fungicides, etc.

In the field of the vectorization of active principles, especially medicinal active principles, there is a need in many cases to:
protect them from degradation (hydrolysis, precipitation at the site, enzymatic digestion, etc.) until they reach their site of action,
and/or control their rate of release so as to maintain a therapeutic level over a defined period,
and/or transport them (with protection) to the site of action.

For these purposes, several types of polymers have been studied and some are even commercially available. Examples which may be mentioned are polymers of the polylactic, polylactic-glycolic, polyoxyethylene-oxypropylene, polyamino acid or polysaccharide type. These polymers constitute starting materials for the manufacture of e.g. mass implants, microparticles, nanoparticles, vesicles, micelles or gels. In addition to the fact that these polymers have to be suitable for the manufacture of such systems, they must also be biocompatible, non-toxic, non-immunogenic and economic and they must be easy to eliminate from the body and/or biodegradable. On this last point, it is further essential that biodegradation in the organism generates non-toxic products.

Another very important point in the development of an associative polymer is its solubility in water. The possibility of solubilizing a large amount of polymer allows the polymer/active principle ratio to be adapted to the desired release profile.

Various patents, patent applications or scientific articles are referred to below in order to illustrate the prior art relating to polymers employed as starting materials for the preparation of AP vectorization systems.

U.S. Pat. No. 4,652,441 describes polylactide microcapsules encapsulating the hormone LH-RH. These microcapsules are produced by preparing a water-in-oil-in-water emulsion and comprise an aqueous inner layer containing the hormone, a substance (gelatin) for fixing the latter, an oily polylactide layer and an aqueous outer layer (polyvinyl alcohol). The AP can be released over a period of more than two weeks after subcutaneous injection.

U.S. Pat. No. 6,153,193 describes compositions based on amphiphilic poly(oxyethylene)-poly(oxypropylene) micelles for the vectorization of anticancer agents such as adriamycin.

Akiyoshi et al. (J. Controlled Release 1998, 54, 313-320) describe pullulans which are rendered hydrophobic by the grafting of cholesterol and form nanoparticles in water. These nanoparticles, which are capable of complexing reversibly with insulin, form stable colloidal suspensions.

U.S. Pat. No. 4,351,337 describes amphiphilic copolyamino acids based on leucine and glutamate which can be used in the form of implants or microparticles for the controlled release of active principles. The latter can be released over a very long period, depending on the rate of degradation of the polymer.

U.S. Pat. No. 4,888,398 describes polymers based on polyglutamate or polyaspartate, and optionally polyleucine, with pendent groups of the alkoxy-carbonylmethyl type randomly located along the polyamino acid chain. These polyamino acids, grafted with side groups, e.g. methoxycarbonylmethyl groups, can be used in the form of prolonged-release biodegradable implants containing an AP.

U.S. Pat. No. 5,904,936 describes nanoparticles obtained from a polyleucine-polyglutamate block polymer which are capable of forming stable colloidal suspensions and of associating spontaneously with biologically active proteins without denaturing them. The latter can then be released in vivo in a controlled manner over a long period.

U.S. Pat. No. 5,449,513 describes amphiphilic block copolymers comprising a polyoxyethylene block and a polyamino acid block, e.g. poly(beta-benzyl-L-aspartate). These polyoxyethylene-polybenzylaspartate polymers form micelles capable of encapsulating hydrophobic active molecules such as adriamycin or indomethacin.

Patent application WO-A-99/61512 describes polylysines and polyornithines functionalized with a hydrophobic group (palmitic acid joined to the polylysine or polyornithine) and a hydrophilic group (polyoxyethylene). In the presence of cholesterol, these polymers, e.g. polylysine grafted with polyoxyethylene and palmitoyl chains, form vesicles capable of encapsulating doxorubicin or DNA. These polymers based on polylysines are cationic in a physiological medium.

U.S. Pat. No. 6,630,171 in the name of the Applicant describes poly(sodium glutamate)-poly(methyl, ethyl, hexadecyl or dodecyl glutamate) block or random polymers capable of forming stable colloidal suspensions and of associating spontaneously with biologically active proteins without denaturing them. The latter can then be released in vivo in a controlled manner over a long period. These amphiphilic linear copolyamino acids are modified by the presence of a hydrophobic alkyl side chain. These alkyl groups are covalently grafted onto the polymer via an ester group.

In the same field, the Applicant has described polyglutamate-based polymers with related designs in several patent applications.

Patent application WO-A-03/104303 describes anionic polyamino acids functionalized with alpha-tocopherol. Patent application WO-A-04/013206 describes anionic polyamino acids containing hydrophobic groups and characterized in that these groups are joined to the polymer via a spacer containing two amide groups, and more precisely via a spacer of the lysine or ornithine type. Unpublished patent application PCT/FR03/03458 describes polyamino acids functionalized with at least one oligoamino acid group based on leucine and/or isoleucine and/or valine and/or phenylalanine.

Patent application WO-A-87/03891 (U.S. Pat. No. 4,892,733) describes polyglutamates or polyaspartates carrying diacid groups of the malonic, succinic or glutaric type bonded to the polyamino acid chain via a spacer of oligopeptide type. The presence of the diacid group makes it possible to fix calcium cations or to form cyclic anhydrides capable of reacting with an active principle. These polymers can be used especially in the form of implants for the slow release of an active principle in vivo.

Thus, although there are a very large number of technical solutions in the prior art which have been developed and proposed for the vectorization of medicinal active principles, it is difficult to respond to all the demands and the situation remains unsatisfactory. More specifically, it has been possible to identify an unsatisfied need for a biodegradable material for producing particles for the vectorization of active principles, which material should be capable of forming an aqueous suspension of vectorization nanoparticles or microparticles suitable for associating reversibly with active principles, and in which one of the desired improvements would be to have the highest possible polymer/active principle ratio.

In this context, one of the essential objects of the present invention is to provide novel amphiphilic linear polyamino acids anionic at animal physiological pH (e.g. in the order of 7.4) which represent an improvement relative to those described in patents or patent applications U.S. Pat. No. 6,630,171, WO-A-03/104303, WO-A-04/013206 and PCT/FR03/03458 (unpublished), especially in terms of the formulation (reversible association) of an active principle such as a therapeutic protein.

Another essential object of the present invention is that these polymers are capable of being used for the vectorization of AP and make it possible optimally to satisfy all or some of the specifications of the specifications sheet, namely, in particular:
capacity:
   easily and economically to form stable aqueous colloidal suspensions,
   easily to associate with numerous active principles,
   and to release these active principles in vivo,
biocompatibility,
biodegradability.

This and other objects are achieved by the present invention, which relates first and foremost to an amphiphilic linear or branched polyamino acid comprising anionic aspartic units and/or glutamic units and containing hydrophobic grafts, all or some of these grafts being anionic or “anionizable”.

It is to the Applicant's credit to have had the idea of functionalizing, in a totally judicious and advantageous manner, biodegradable polyamino acids of the polyAsp and/or polyGlu type with hydrophobic grafts carrying anionic charges and/or with “anionizable” hydrophobic grafts.

In other words, the invention relates to a polyamino acid comprising aspartic units and/or glutamic units, some of which carry one or more identical or different hydrophobic grafts,

characterized in that:
the hydrophobic grafts have general formula (I) below:

—X-(GH)—Y, (I)
in which:
[symbol] —X— is a link unit between the polyamino acid chain and -GH—Y;
[symbol] -GH— is a hydrophobic group; and
[symbol] —Y is a group having at least one anionic charge and/or one or more identical or different ionizable groups, each of which is capable of giving rise to at least one anionic charge, and
-GH— is devoid of an alpha-amino acid residue.

Advantageously, —Y can be devoid of diacid groups of the malonic, succinic or glutaric type.

This polyamino acid comprises main chains (or skeletons) consisting at least in part of aspartic units and/or glutamic units. In practice, the polyamino acid is essentially formed of these monomeric aspartic and/or glutamic units.

An Asp or Glu unit can be grafted with one or two (in practice only one) anionic or “anionizable” hydrophobic grafts.

These novel amphiphilic polyGlu and/or polyAsp polymers have proved particularly suitable especially for the vectorization of proteins. Surprisingly, it has been found that these novel polymers can be dissolved in aqueous solution at much higher concentrations than analogous polymers of the prior art. This property is a great asset for being able to produce, as required, a formulation with a higher polymer/active principle ratio.

As defined in the invention:
the term “polyamino acid” covers on the one hand PAA containing a single type of “amino acid” unit (e.g. either Glu (glutamic or glutamate) units or Asp (aspartic or aspartate) units, or a copolyamino acid (containing a mixture of Glu and Asp amino acid units in a concatenation of random, gradient or block type), and on the other hand both oligoamino acids comprising from 2 to 20 “amino acid” units and polyamino acids comprising more than 20 “amino acid” units;
the term “amino acid unit” relates to a monomeric or non-monomeric unit formed of a skeleton of a given amino acid, regardless of what the substituents might be, provided they do not modify the nature of the amino acid in question.

These polymers have surprising properties of fluidity, association and/or encapsulation with one or more active principles, compared with analogous products.

As defined in the invention and throughout the present disclosure, the terms “association” or “associate” employed to qualify the relationships between one or more active principles and the polyamino acids denote in particular that the active principle(s) is (are) bonded to the polyamino acid(s) especially by a weak bond, e.g. by ionic bonding and/or hydrophobic contact, and/or are encapsulated by the polyamino acid(s).

Another considerable advantage of the polyamino acids according to the invention is that they are easily enzymatically degraded or degradable to non-toxic catabolites/metabolites (amino acids).

The link unit —X— of the hydrophobic graft (I), —X-GH—Y, can also be referred to as a spacer, making it possible to join the hydrophobic group GH to a main chain of the polyamino acid. —X— can comprise e.g. at least one direct covalent bond and/or at least one amide linkage and/or at least one ester linkage. For example, —X— can be a unit of the type belonging to the group comprising “amino acid” units different from the constituent monomeric unit of the polyamino acid, amino alcohol derivatives, diamine derivatives, diol derivatives and hydroxy acid derivatives.

The hydrophobic grafts (I), —X-GH—Y, can be grafted onto the polyamino acid chain in various ways. It is possible:
a) either firstly to graft —X— onto the polyamino acid chain or use the pendent reactive groups of the polyamino acid main chain as a spacer —X—, then to fix -GH— to the —X— of the polyamino acid chain, and finally to fix —Y to the GH,
b) or firstly to graft —X— onto the polyamino acid chain or use the pendent reactive groups of the polyamino acid main chain as a spacer —X—, and then to preassemble a block -GH—Y or use a block -GH—Y pre-existing as such, said -GH—Y then being grafted onto the —X— of the polyamino acid chain,
c) or to preassemble a block —X-GH—Y, which is then grafted onto the polyamino acid chain, or to preassemble a block —X-GH or use a block —X-GH pre-existing as such, said —X-GH then being grafted onto the polyamino acid chain, and —Y finally being grafted to the GH.

Method b) is preferred in practice, using the pendent reactive groups of the polyamino acid main chain as a spacer —X— and using a block -GH—Y pre-existing as such, said -GH—Y then being grafted onto the —X— of the polyamino acid chain.

The grafting of the GH is explained in greater detail below in the description of the process for obtaining the polyamino acids according to the invention.

Conventionally, the precursors of the -GH—Y or -GH are selected e.g. from the group comprising alcohols and amines, these compounds being easily functionalizable by those skilled in the art.

In one preferred variant of the invention, the polyamino acid is characterized in that —X— and/or —Y is (are) devoid of one or more alpha-amino acid residues. Very particularly preferably, —X—, -GH— and —Y are devoid of alpha-amino acids.

According to one preferred characteristic, the hydrophobic group GH of the hydrophobic graft contains from 8 to 30 carbon atoms.

Preferably, the ionizable group(s) of —Y capable of giving rise to at least one anionic charge is (are) selected from the group comprising the carboxylic/carboxylate group, the sulfonic/sulfonate group, the sulfuric/sulfate group and the phosphoric/phosphate group.

Very particularly preferably, the hydrophobic groups GH are selected from the group comprising:
linear or branched C8 to C30 alkyls which can optionally contain at least one unit of unsaturation and/or at least one heteroatom,
C8 to C30 alkylaryls or arylalkyls which can optionally contain at least one unit of unsaturation and/or at least one heteroatom, and
C8 to C30 (poly)cyclics which can optionally contain at least one unit of unsaturation and/or at least one heteroatom.

The polyamino acid according to the invention is preferably one of those comprising alpha-L-glutamate and/or alpha-L-glutamic units or alpha-L-aspartate and/or alpha-L-aspartic units.

More precisely, the polyamino acids according to the present invention are e.g. homooligomers or homopolymers comprising alpha-L-glutamate and/or alpha-L-glutamic units or alpha-L-aspartate and/or alpha-L-aspartic units.

Particularly preferably, the polyamino acids according to the invention have general formula (II) below:

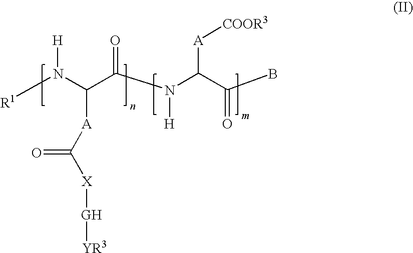

in which:
R¹ is H, a linear C2 to C10 or branched C3 to C10 acyl group, or pyroglutamate;
A is independently —CH₂— (aspartic unit) or —CH₂—CH₂— (glutamic unit);
B is:
   OR³, R³ being as defined below,
   a group NHR², in which R² is H, a linear C2 to C10 or branched C3 to C10 alkyl, or benzyl,
   a terminal amino acid unit which is bonded by the nitrogen and whose acid group(s) is (are) optionally modified by an amine or alcohol respectively defined as NHR² and OR²;
R³ is H or a cationic entity preferably selected from the group comprising:
   metal cations advantageously selected from the subgroup comprising sodium, potassium, calcium and magnesium,
   organic cations advantageously selected from the subgroup comprising:
      cations based on amine,
      cations based on oligoamine,
      cations based on polyamine (polyethylenimine being particularly preferred), and
      cations based on one or more amino acid residues advantageously selected from the class comprising cations based on lysine or arginine,
   and cationic polyamino acids advantageously selected from the subgroup comprising polylysine and oligolysine;
—X— is a link unit —O—, —NH— or —N-alkyl- (C1 to C5), an amino acid residue (preferably natural), a diol, a diamine, an amino alcohol or a hydroxy acid containing from 1 to 6 carbon atoms;
-GH— is a hydrophobic group containing 8 to 30 carbon atoms and preferably selected from the group comprising:
   linear or branched C8 to C30 alkyls which can optionally contain at least one unit of unsaturation and/or at least one heteroatom (preferably O and/or N and/or S),
   C8 to C30 alkylaryls or arylalkyls which can optionally contain at least one unit of unsaturation and/or at least one heteroatom (preferably O and/or N and/or S), and
   C8 to C30 (poly)cyclics which can optionally contain at least one unit of unsaturation and/or at least one heteroatom (preferably O and/or N and/or S);
—Y is:
   an “anionizable” group consisting of a carboxylic, sulfonic, sulfuric or phosphoric group (R³ is H), or
   Y is an anionic group preferably consisting of a carboxylate, sulfonate, sulfate or phosphate group (R³ is different from H);
n/(n+m) is defined as the molar grafting rate and varies from 0.5 to 100 mol %; and
n+m varies from 3 to 1000, preferably between 30 and 500.

In practice, at least one of the hydrophobic grafts of formula (I), —X-GH—Y, is selected e.g. from the group comprising the following species:

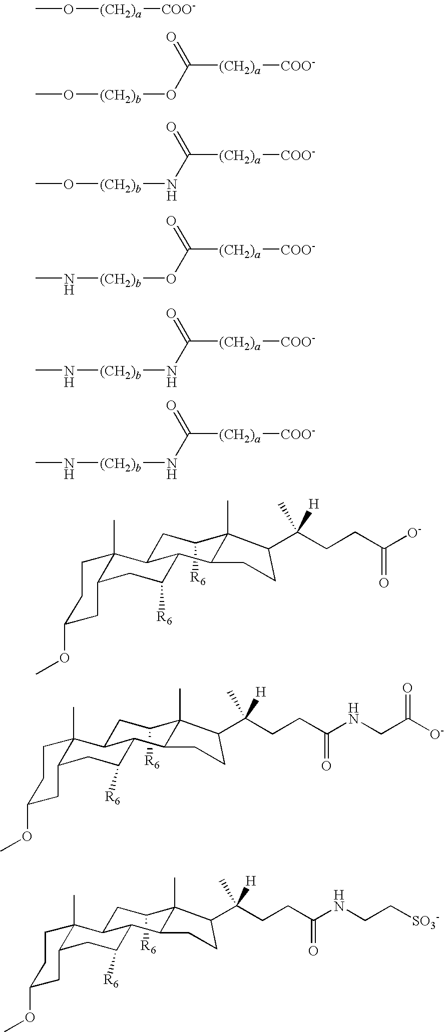

In these structures:

a is between 7 and 19,

b is between 2 and 4, and

R₆ is H or OH.

In one variant of the invention, the polyamino acids to which it relates not only carry hydrophobic grafts having an anionic charge Y or a group Y ionizable to an anion, but can also carry at least one non-ionizable hydrophobic graft. In particular, the polyamino acid can carry at least one hydrophobic graft of formula (III), —X′-GH′, which is devoid of an ionized or ionizable group and in which —X′— and -GH′ are as defined above for —X— and -GH—.

Thus the non-ionized or non-ionizable group GH′ can be derived e.g. from a group selected from the group comprising the following species: octanol, dodecanol, tetradecanol, hexadecanol, octadecanol, oleyl alcohol, tocopherol and cholesterol.

In a first embodiment of the invention, the main chains of the polyamino acids are alpha-L-glutamate or alpha-L-glutamic homopolymers.

In a second embodiment of the invention, the main chains of the polyamino acids are alpha-L-aspartate or alpha-L-aspartic homopolymers.

In a third embodiment of the invention, the main chains of the polyamino acids are alpha-L-aspartate/alpha-L-glutamate or alpha-L-aspartic/alpha-L-glutamic copolymers.

Advantageously, the distribution of the aspartic and/or glutamic units of the polyamino acid main chain is such that the resulting polymers are either random, or of the block type, or of the multiblock type.

According to another definition, the polyamino acids according to the invention have a molecular weight of between 2000 and 200,000 g/mol, preferably of between 5000 and 50,000 g/mol.

It is furthermore preferable for the molar grafting rate of hydrophobic grafts rate of hydrophobic grafts of the polyamino acids according to the invention to be between 2 and 100%, preferably between 5 and 50%.

In some variants, the polyamino acids according to the invention can be linear or branched.

In other variants, the polyamino acids according to the invention can carry at least one graft of the polyethylene glycol type bonded to a glutamate and/or aspartate unit.

Naturally, the invention also covers mixtures of polyamino acids as defined above.

Remarkably, the polyamino acids of the invention can be used in several ways, depending on the nature of the hydrophobic groups and the degree of polymerization of the polyamino acid. The methods of forming a polymer for the encapsulation of an active principle in the various forms envisaged by the invention are known to those skilled in the art. For further details, reference may be made e.g. to the following few references of particular pertinence:
“Microspheres, Microcapsules and Liposomes; vol. 1. Preparation and chemical applications” Ed. R. Arshady, Citus Books 1999. ISBN: 0-9532187-1-6.
“Sustained-Release Injectable Products” Ed. J. Senior and M. Radomsky, Interpharm Press 2000. ISBN: 1-57491-101-5.
“Colloidal Drug Delivery Systems” Ed. J. Kreuter, Marcel Dekker, Inc. 1994. ISBN: 0-8247-9214-9.
“Handbook of Pharmaceutical Controlled Release Technology” Ed. D. L. Wise, Marcel Dekker, Inc. 2000. ISBN: 0-8247-0369-3.

These polyamino acids are also extremely valuable in that, depending on the length of the polymer (degree of polymerization) and the nature of the hydrophobic groups, they disperse in water at pH 7.4 (e.g. with a phosphate buffer) to give colloidal solutions or suspensions or structured or non-structured gels, depending on the polymer concentration. Furthermore, the polyamino acids (in particulate or non-particulate form) can encapsulate or easily associate with active principles such as proteins, peptides or small molecules. The preferred forming method is the one described in U.S. Pat. No. 6,630,171 in the name of the Applicant, which consists in dispersing the polymer in water and incubating the solution in the presence of an active principle (AP). This colloidal solution of vectorization particles consisting of the polyamino acids according to the invention can then be passed through a 0.2 μm filter and then injected directly into a patient.

When the hydrophilic/hydrophobic ratio decreases, the polymer can form microparticles capable of associating or encapsulating AP. In this context the microparticles can be formed by cosolubilizing the AP and the polymer in an appropriate organic solvent and then precipitating the mixture in water. The particles are subsequently recovered by filtration and can then be used for oral administration (in the form of gelatin capsules, in compacted and/or coated form, or else in the form of a dispersion in an oil) or for parenteral administration after redispersion in water.

In one variant the polymer can be solubilized in a biocompatible solvent such as N-methylpyrrolidone, or an appropriate oil such as Mygliol®, and then injected by the intramuscular or subcutaneous route or into a tumor. Diffusion of the solvent or oil leads to precipitation of the polymer at the injection site and thus forms a depot. These depots then ensure a controlled release by diffusion and/or by erosion and/or by hydrolytic or enzymatic degradation of the polymer.

Independently of the fact that the microparticulate form of the polyamino acid according to the invention is preferred, the polymers of the invention, in neutral or ionized form, can more generally be used by themselves or in a liquid, solid or gel composition and in an aqueous or organic medium.

It should be understood that the polymer based on polyamino acids contains groups which are either neutral or ionized, depending on the pH and the composition. In aqueous solution the countercation can be a metal cation such as sodium, calcium or magnesium, or an organic cation such as triethanolamine, tris(hydroxymethyl)aminomethane or a polyamine like polyethylenimine.

The polymers of the invention are obtained e.g. by methods known to those skilled in the art. The random polyamino acids can be obtained by grafting the hydrophobic graft formed by a -GH or a -GH—Y, previously functionalized with a link unit —X— containing e.g. at least one amino acid, directly onto the polymer by means of a conventional coupling reaction. The link unit —X— can initially belong to the main chain of the polyamino acid. The block or multiblock polyamino acids can be obtained by sequential polymerization of the corresponding amino acid N-carboxy anhydrides (NCA).

For example, a homopolyglutamate or homopolyaspartate polyamino acid or a block, multiblock, random or linear glutamate/aspartate copolymer is prepared by conventional methods.

To obtain a polyamino acid of the alpha type, the most common technique is based on the polymerization of amino acid N-carboxy anhydrides (NCA), which is described e.g. in the article “Biopolymers” 1976, 15, 1869, and in the work by H. R. Kricheldorf entitled “Alpha-amino acid N-carboxy anhydrides and related heterocycles”, Springer Verlag (1987). The NCA derivatives are preferably NCA-O-Me, NCA-O-Et or NCA-O-Bz derivatives (Me=methyl, Et=ethyl and Bz=benzyl). The polymers are then hydrolyzed under appropriate conditions to give the polymer in its acid form. These methods are based on the description given in patent FR-A-2 801 226 in the name of the Applicant. A number of polymers that can be used according to the invention, e.g. of the poly(alpha-L-aspartic), poly(alpha-L-glutamic), poly(alpha-D-glutamic) and poly(gamma-L-glutamic) types of variable molecular weights, are commercially available. The polyaspartic polymer of the alpha-beta type is obtained by the condensation of aspartic acid (to give a polysuccinimide) followed by basic hydrolysis (cf. Tomida et al., Polymer 1997, 38, 4733-36).

Still by way of a non-limiting illustration, coupling of the hydrophobic graft carrying GH with an acid group of the polymer (link unit —X—) can easily be effected e.g. by reacting the polyamino acid in the presence of a carbodiimide as coupling agent, and optionally a catalyst such as 4-dimethylaminopyridine, in an appropriate solvent such as dimethylformamide (DMF), N-methylpyrrolidone (NMP) or dimethyl sulfoxide (DMSO). The carbodiimide is e.g. dicyclohexyl-carbodiimide or diisopropylcarbodiimide. The grafting rate is controlled chemically by the stoichiometry of the constituents and reactants or by the reaction time. The hydrophobic grafts functionalized with an amino acid (spacer) are obtained by conventional peptide coupling or by direct condensation under acid catalysis. These techniques are well known to those skilled in the art.

According to another of its features, the invention relates to a pharmaceutical, cosmetic, dietetic or phytosanitary composition comprising at least one polyamino acid as defined above and optionally at least one active principle, which can be a therapeutic, cosmetic, dietetic or phytosanitary active principle in particular.

According to one valuable provision of the invention, the active principle is associated with the polyamino acid(s) by one or more bonds other than covalent chemical bonds.

The techniques of associating one or more AP with the grafted polyamino acids according to the invention are described in particular in patent U.S. Pat. No. 6,630,171. They consist in incorporating at least one active principle into the liquid medium containing particles in order to give a colloidal suspension laden or associated with one or more active principles AP. This incorporation, which results in the AP being trapped by the particles, can be effected in the following manner:
introduction of AP into aqueous solution, followed by addition of the polymer, either in the form of a colloidal suspension or in the form of isolated particles (lyophilizate or precipitate); or
addition of AP, either in solution or in the pure or preformulated state, to a colloidal suspension of particles, optionally prepared for immediate use by dispersion of dry vectorization particles (VP) in an appropriate solvent such as water.

Preferably, the active principle is a protein, a glycoprotein, a protein bonded to one or more polyalkylene glycol chains (preferably polyethylene glycol (PEG) chains: “PEGylated protein”), a polysaccharide, a liposaccharide, an oligonucleotide, a polynucleotide or a peptide.

In one variant the active principle is a “small” hydrophobic, hydrophilic or amphiphilic organic molecule.

As defined in the present disclosure, a “small” molecule is especially a small non-protein molecule.

The following may be mentioned as examples of AP that can be associated with the polyamino acids according to the invention, whether or not they are in the form of nanoparticles or microparticles:
proteins such as insulin, interferons, growth hormones, interleukins, erythropoietin or cytokines;
peptides such as leuprolide or cyclosporin;
small molecules such as those belonging to the anthracycline, taxoid or camptothecin family;
and mixtures thereof.

In one embodiment the composition of the invention is in the form of a gel, a solution, a suspension, an emulsion, micelles, nanoparticles, microparticles, an implant, a powder or a film.

In one of its particularly preferred forms, the composition, whether or not laden with active principle(s), is a stable colloidal suspension of nanoparticles and/or microparticles and/or micelles of polyamino acids in an aqueous phase.

In another embodiment the composition of the invention is in the form of a solution in a biocompatible solvent and can be injected by the subcutaneous or intramuscular route or into a tumor.

In another embodiment the composition can optionally contain an excipient to adjust the pH and/or the osmolarity, and/or to improve the stability (antioxidants) and/or as an antimicrobial. These excipients are well known to those skilled in the art (refer to the work entitled Injectable Drug Development, P. K. Gupta et al., Interpharm Press, Denver, Colo. 1999).

If the composition according to the invention is a pharmaceutical composition, it can be administered by the oral, parenteral, nasal, vaginal, ocular, subcutaneous, intravenous, intramuscular, intradermal, intraperitoneal, intracerebral or buccal route.

It is also possible to envisage a composition in the form of a solution in a biocompatible solvent that can be injected by the subcutaneous or intramuscular route or into a tumor.

In another variant the composition according to the invention is formulated in such a way that it is capable of forming a depot at the injection site.

The invention further relates to compositions which comprise polyamino acids according to the invention and active principles and which can be used for the preparation of:
drugs, particularly for administration by the oral, nasal, vaginal, ocular, subcutaneous, intravenous, intramuscular, intradermal, intraperitoneal or intracerebral route, it being possible in particular for the active principles of these drugs to be proteins, glycoproteins, proteins bonded to one or more polyalkylene glycol chains {e.g. polyethylene glycol (PEG) chains, in which case the term “PEGylated” proteins is used}, peptides, polysaccharides, liposaccharides, oligonucleotides, polynucleotides and small hydrophobic, hydrophilic or amphiphilic organic molecules;
and/or nutriments;
and/or cosmetic or phytosanitary products.

According to yet another of its features, the invention relates to a process for the preparation of:
drugs, particularly for administration by the oral, nasal, vaginal, ocular, subcutaneous, intravenous, intramuscular, intradermal, intraperitoneal or intracerebral route, it being possible in particular for the active principles of these drugs to be proteins, glycoproteins, proteins bonded to one or more polyalkylene glycol chains {e.g. polyethylene glycol (PEG) chains, in which case the term “PEGylated” proteins is used}, peptides, polysaccharides, liposaccharides, oligonucleotides, polynucleotides and small hydrophobic, hydrophilic or amphiphilic organic molecules;
and/or nutriments;
and/or cosmetic or phytosanitary products,
   said process being characterized in that it consists essentially in using at least one polyamino acid as defined above and/or the composition also described above.

The invention further relates to a method of therapeutic treatment that consists essentially in administering the composition as described in the present disclosure by the oral, parenteral, nasal, vaginal, ocular, subcutaneous, intravenous, intramuscular, intradermal, intraperitoneal, intracerebral or buccal route.

In one particular variant of the invention, said method of therapeutic treatment consists essentially in introducing the composition as described above into solution in a biocompatible solvent and then injecting it by the subcutaneous or intramuscular route or into a tumor, preferably in such a way that it forms a depot at the injection site.

The invention will be better understood and its advantages and variants will become clearly apparent from the Examples below, which describe the synthesis of the polyamino acids, their conversion to an AP vectorization system (stable aqueous colloidal suspension) and the demonstration of the ability of such a system to associate with a protein to form pharmaceutical compositions.

### EXAMPLE 1

### Synthesis of the Polymer pGluLC

Synthesis of a Polyglutamate (Whose Reactive Carboxylate Side-Groups Act as a Link Unit —X—) Grafted with a Hydrophobic Group GH Derived from Lithocholic Acid

### 1/Structure of the Precursor of GH:

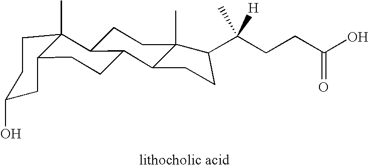

### Step 1: Grafting of the Acids

The alpha-L-polyglutamic polymer (having a molecular weight equivalent to about 12,000 g/mol, relative to a polyoxyethylene standard) is obtained by the polymerization of monomers consisting of N-carboxy anhydride derivatives of methyl glutamate: NCAGluOMe. This polymerization is followed by hydrolysis, as described in patent application FR-A-2 801 226.

In a 500 ml reactor under a stream of nitrogen, 5 g of pGluOH are solubilized in 90 ml of DMF at 80° C. The solution is stirred for 18 h at 80° C. and then cooled to 15° C. After the addition of 0.6 g of diisopropylcarbodiimide (DIPC), the reaction mixture is stirred for 30 min and a solution of 1.75 g of lithocholic acid (LC) in 5 ml of DMF and a solution of 0.142 g of 4-dimethylaminopyridine (DMAP) in 3 ml of DMF are then added in succession. The reaction mixture is stirred for 18 h at 15° C., 5 ml of 1 N HCl are added and the polymer is then precipitated by pouring the reaction mixture dropwise into 400 ml of acidified aqueous sodium chloride solution (60 g of NaCl, pH=2, HCl) and 200 ml of diisopropyl ether. After filtration on a glass frit, the solid obtained is washed with 0.1 N HCl solution and then with a water/ethyl acetate mixture. Five grams of a white solid are obtained after drying in a vacuum oven.

The grafting rate measured by proton NMR in TFA-d is 7.2%. The Mn (determined by GPC in NMP at 70° C.) is 34,800 g/mol in PMMA equivalents.

### Step 2: Neutralization

Two grams of the polymer of step 1 are suspended in 200 ml of demineralized water. 0.1 N sodium hydroxide solution is added dropwise until the solid has totally dissolved, care being taken not to exceed pH 9. After neutralization, the pH is adjusted to 7.4 with 0.1 N HCl solution. The Mn (determined by aqueous GPC) is 16,600 g/mol in POE equivalents.

### EXAMPLE 2

### Synthesis of the Polymer pGluHLA

Synthesis of a Polyglutamate (Whose Reactive Carboxylate Side-Groups Act as a Link Unit —X—) Grafted with GH Derived from 12-Hydroxylauric Acid (HLA)

### 1/Structure of the Precursor of GH:

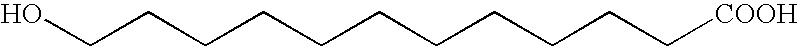

### Step 1: Grafting of the Acids

In a 500 ml reactor under a stream of nitrogen, 10 g of pGluOH (idem Example 1) are solubilized in 180 ml of DMF at 80° C. and the solution is stirred for 18 h at this temperature. The reaction mixture is cooled to 15° C., 3.91 g of DIPC are added and the mixture is stirred for 30 min. A solution of 5.87 g of HLA in 15 ml of DMF and a solution of 0.57 g of DMAP in 5 ml of DMF are then added in succession. The mixture is stirred at 15° C. for 6 h, 9 ml of 1 N HCl are added and the polymer is then precipitated by pouring the reaction mixture dropwise into 1 l of acidified water (HCl) of pH 2. The white solid formed is filtered off on a glass frit and then washed successively with 0.1 N HCl solution, with water, with ethyl acetate and finally with diisopropyl ether. After drying in a vacuum oven, 12.6 g of a white solid are isolated.

The grafting rate measured by proton NMR in TFA-d is 27.4%. The Mn (determined by GPC in NMP at 70° C.) is 38,000 g/mol in PMMA equivalents.

### Step 2: Neutralization

4 g of the polymer obtained in step 1 are suspended in 200 ml of demineralized water. The suspension is stirred vigorously and 0.1 N sodium hydroxide solution is added dropwise until the solid has totally dissolved, care being taken not to exceed pH 8. After neutralization, the pH is adjusted to 7.4 with 0.1 N HCl solution. The Mn (determined by aqueous GPC) is 18,600 g/mol in POE equivalents.

### EXAMPLE 3

### Synthesis of the Polymer pGluHLA-T

Synthesis of a Polyglutamate (Whose Reactive Carboxylate Side-Groups Act as a Link Unit —X—) Grafted with GH Derived from 12-Hydroxylauric Acid (HLA) and GH′ Derived from Alpha-Tocopherol (T)

### 1/Structures of the Precursors of GH:

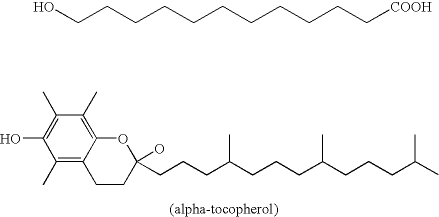

Step 1: 1st Grafting of the Acids (with D,L-Alpha-Tocopherol of Synthetic Origin)

In a 500 ml reactor under a stream of nitrogen, 5 g of pGluOH (idem Example 1) and 47 mg of DMAP are solubilized in 90 ml of DMF at 80° C. and the solution is stirred for 18 h at this temperature. The reaction mixture is cooled to 15° C., a solution of 1.67 g of D,L-alpha-tocopherol in 5 ml of DMF, a solution of 940 mg of DMAP in 5 ml of DMF and then 780 mg of DIPC are added in succession. The mixture is stirred at 15° C. for 3 h 30 min, 5 ml of 1 N HCl are added and the polymer is then precipitated by pouring the reaction mixture dropwise into 400 ml of acidified aqueous sodium chloride solution (60 g of NaCl, pH=2, HCl) and 200 ml of diisopropyl ether. The white solid formed is filtered off on a glass frit and then washed successively 3 times with a mixture of 300 ml of water and 200 ml of diisopropyl ether and then twice with 300 ml of diisopropyl ether. After drying in a vacuum oven, 5.1 g of a white solid are isolated.

The grafting rate measured by proton NMR in TFA-d is 9.5%. The Mn (determined by GPC in NMP at 70° C.) is 39,100 g/mol in PMMA equivalents.

Step 2: 2nd Grafting of the Acids (with HLA)

In a 500 ml reactor under a stream of nitrogen, 5 g of the polymer obtained in step 1 are solubilized in 90 ml of DMF at 80° C. and the solution is stirred for 18 h at this temperature. The reaction mixture is cooled to 15° C., 1.95 g of DIPC are added and the mixture is stirred for 30 min. A solution of 3.35 g of HLA in 5 ml of DMF and a solution of 280 mg of DMAP in 5 ml of DMF are then added in succession. The mixture is stirred at 15° C. for 2 h and then at 20° C. for 4 h. 5 ml of 1 N HCl are added and the polymer is then precipitated by pouring the reaction mixture dropwise into 600 ml of acidified water (HCl) of pH 2. The white solid formed is filtered off on a glass frit, resolubilized in 100 ml of DMF and then precipitated again by pouring the reaction mixture dropwise into 600 ml of acidified water (HCl) of pH 2. The white solid formed is filtered off on a glass frit and then washed successively with 0.1 N HCl solution, with water and finally with diisopropyl ether. After drying in a vacuum oven, 5.6 g of a white solid are isolated.

The grafting rate measured by proton NMR in TFA-d is 35%. The Mn (determined by GPC in NMP at 70° C.) is 43,000 g/mol in PMMA equivalents.

### Step 3: Neutralization

2 g of the polymer obtained in step 2 are suspended in 200 ml of demineralized water. The suspension is stirred vigorously and 1 N sodium hydroxide solution is added dropwise until the solid has totally dissolved, care being taken not to exceed pH 8. After neutralization, the pH is adjusted to 7.4 with 0.1 N HCl solution. The Mn (determined by aqueous GPC) is 12,900 g/mol in POE equivalents.

### EXAMPLE 4

### Studies of the Solubility and Viscosity Properties

To compare the properties and demonstrate the invention, 3 polymers of analogous structures were synthesized according to the same procedures. These polymers have the following grafts (without any ionic charge):
Cl: cholesterol (CHOL)
C2: n-dodecanol (OC12)
C3: alpha-tocopherol (T)

The viscosity of the polymer is measured as a function of concentration at pH 7.4 and at an osmolality of 300 mOsmol. The limiting aggregation concentration Cη (g/l), i.e. the concentration beyond which the viscosity increases very rapidly, is then measured. The results are collated in the Table below.

TABLE 1

| Example | Polymer | mol % of the graft | Cη (g/l) |
|---|---|---|---|
| Example 1 | pGluLC | 7% | 90 g/l |
| Example 2 | pGluHLA | 27% | 150 g/l |
| Example 3 | pGluHLA-T | HLA: 35%, T: 10% | >40 g/l |
| C1 | pGluChol | 5% | 35 g/l |
| C2 | pGluOC12 | 20% | 30 g/l |
| C3 | pGluT | 7% | 20 g/l |

C1 and C2: polyglutamate according to patent US-B-6,630,171
C3: polyglutamate according to patent WO-A-03/104303

Comparison of the viscosities, illustrated by the Cη values, shows that it is much easier to obtain a concentrated solution with the polymers of the invention. This property therefore makes it possible to prepare formulations with high polymer concentrations, thereby making it possible to increase the polymer/active principle ratio, while at the same time ensuring a good injectability.

### EXAMPLE 5

### Study of Association with Insulin

An aqueous solution of pH 7.4 containing a defined amount of polymer per milliliter and 200 IU of insulin (7.4 mg) is prepared. The solutions are incubated for two hours at room temperature and the free insulin is separated from the associated insulin by ultrafiltration (cut-off at 100 kDa, 15 minutes under 10,000 G at 18° C.). The free insulin recovered from the filtrate is then measured quantitatively by HPLC (high performance liquid chromatography) and the amount of associated insulin is deduced. The results are given in Table 2 below.

TABLE 2

| Polymer | Polymer concentration | % association |
|---|---|---|
| Ex. 1 | 50 mg/ml | 82% |
| Ex. 3 | 50 mg/ml | 51% |

The results demonstrate that the polymers of the invention are capable of associating insulin to give colloidal suspensions with a size in excess of 100 kDa, and the insulin association rates are very high. The association capacity of these polymers makes them suitable for use as vectorization agents.

1. Polyamino acid comprising aspartic units and/or glutamic units, some of which carry one or more identical or different hydrophobic grafts, characterized in that:
the hydrophobic grafts have general formula (I) below:
—X-(GH)—Y, (I)
in which:
—X— is a link. unit between the polyamino acid chain and
-GH—Y;
-GH— is a hydrophobic group; and
—Y is a group having at least one anionic charge and/or one or more identical or different ionizable groups, each of which is capable of giving rise to at least one anionic charge, and
-GH— is devoid of an alpha-amino acid residue.

2. Polyamino acid according to claim 1, characterized in that —X— and/or —Y are devoid of alpha-amino acid residues.

3. Polyamino acid according to claim 1, characterized in that at least one hydrophobic group -GH— contains from 8 to 30 carbon atoms.

4. Polyamino acid according to claim 1, characterized in that the ionizable group(s) of —Y capable of giving rise to at least one anionic charge is selected from the group comprising the carboxylic/carboxylate group, the sulfonic/sulfonate group, the sulfuric/sulfate group and the phosphoric/phosphate group.

5. Polyamino acid according to claim 1, characterized in that at least one hydrophobic group GH is selected from the group comprising:
linear or branched C8 to C30 alkyls which can optionally contain at least one unit of unsaturation and/or at least one heteroatom,
C8 to C30 alkylaryls or arylalkyls which can optionally contain at least one unit of unsaturation and/or at least one heteroatom, and
C8 to C30 (poly)cyclics which can optionally contain at least one unit of unsaturation and/or at least one heteroatom.

6. Polyamino acid according to claim 1, characterized in that its main chain comprises alpha-L-glutamate and/or alpha-L-glutamic units or alpha-L-aspartate and/or alpha-L-aspartic units.

7. Polyamino acid according to claim 1, characterized in that it has general formula (II) below: 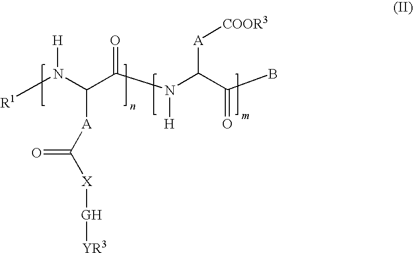
in which:
R¹ is H, a linear C2 to CIO or branched C3 to C1O acyl group, or pyroglutamate;
A is independently —CH₂— (aspartic unit) or CH₂—CH₂— (glutamic unit);
B is:
OR³, R³ being as defined below,
a group NHR², in which R² is H, a linear C2 to CIO or branched C3 to CIO alkyl, or benzyl,
a terminal amino acid unit which is bonded by the nitrogen and whose acid group(s) is (are) optionally modified by an amine or alcohol respectively defined as NHR² and OR²;
R³ is H or a cationic entity preferably selected from the group comprising:
metal cations advantageously selected from the subgroup comprising sodium, potassium, calcium and magnesium;
organic cations advantageously selected from the subgroup comprising:
cations based on amine,
cations based on oligoamine,
cations based on polyamine (polyethylenimine being particularly preferred), and
cations based on one or more amino acid residues advantageously selected from the class comprising cations based on lysine or arginine,
and cationic polyamino acids advantageously selected from the subgroup comprising polylysine and oligo lysine;
—X— is a link unit —O—, —NH— or —N-alkyl- (C1 to C5), an amino acid residue (preferably natural), a diol, a diamine, an amino alcohol or a hydroxy acid;
-GH— is a hydrophobic group containing 8 to 30 carbon atoms and preferably selected from the group comprising:
linear or branched C8 to C30 alkyls which can optionally contain at least one unit of unsaturation and/or at least one heteroatom (preferably O and/or N and/or S),
C8 to C30 alkylaryls or arylalkyls which can optionally contain at least one unit of unsaturation and/or at least one heteroatom (preferably O and/or N and/or S), and
C8 to C30 (poly)cyclics which can optionally contain at least one unit of unsaturation and/or at least one heteroatom (preferably O and/or N and/or S);
—Y is:
an “anionizable” group consisting of a carboxylic, sulfonic, sulfuric or phosphoric group (R³ is H), or
Y is an anionic group preferably consisting of a carboxylate, sulfonate, sulfate or phosphate group (R³ is different from H);
n/(n+m) is defined as the molar grafting rate and varies from 0.5 to 100 mol %; and
n+m varies from 3 to 1000, preferably between 30 and 500.

8. Polyamino acid according to claim 1, characterized in that at least one of the hydrophobic grafts of formula (I), —X-GH—Y, is a radical selected from the group comprising the following species: 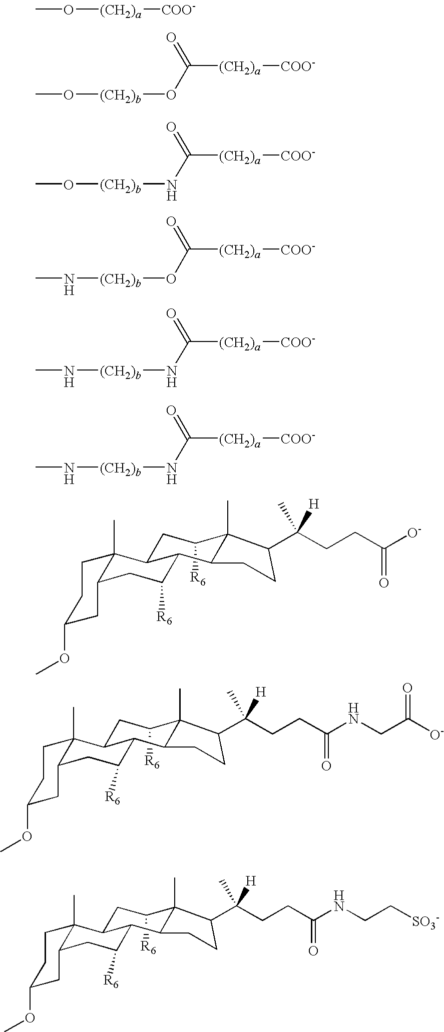
in which:
a is between 7 and 19,
b is between 2 and 4, and
R₆ is H or OH.

9. Polyamino acid according to claim 1, characterized in that it carries at least one hydrophobic graft of formula (III), —X′-GH′, which is devoid of an ionized or ionizable group and in which —X′— and GH′ are as defined for —X— and -GH.

10. Polyamino acid according to claim 9, characterized in that the hydrophobic group GH′ is a derivative of a group selected from the following species:
octanol, dodecanol, tetradecanol, hexadecanol, octadecanol, oleyl alcohol, tocopherol and cholesterol.

11. Polyamino acid according to claim 1, characterized in that its molecular weight is between 2000 and 200,000 g/mol, preferably between 5000 and 50,000 g/mol.

12. Polyamino acid according to claim 1, characterized in that it carries at least one graft of the polyalkylene glycol (preferably polyethylene glycol) type bonded to a glutamate and/or aspartate unit.

13. Pharmaceutical, cosmetic, dietetic or phytosanitary composition comprising at least one polyamino acid according to claim 1.

14. Composition according to claim 13, characterized in that it comprises at least one active principle.

15. Composition according to claim 14, characterized in that the active principle is associated with the polyamino acid(s) by one or more bonds other than covalent chemical bonds.

16. Composition according to claim 14, characterized in that the active principle is a protein, a glycoprotein, a protein bonded to one or more polyalkylene glycol chains, a polysaccharide, a liposaccharide, an oligonucleotide, a polynucleotide or a peptide.

17. Composition according to claim 14, characterized in that the active principle is a small hydrophobic, hydrophilic or amphiphilic organic molecule.

18. Composition according to 13, characterized in that it can be administered by the oral, parenteral, nasal, vaginal, ocular, subcutaneous, intravenous, intramuscular, intradermal, intraperitoneal, intracerebral or buccal route.

19. Composition according to claim 13, characterized in that it is in the form of a gel, a solution, an emulsion, micelles, nanoparticles, microparticles, a powder or a film.

20. Composition according to claim 13, characterized in that it is a colloidal suspension of nanoparticles and/or microparticles and/or micelles of polyamino acids in an aqueous phase.

21. Composition according to claim 13, characterized in that it is in the form of a solution in a biocompatible solvent and in that it can be injected by the subcutaneous or intramuscular route or into a tumor.

22. Composition according to claim 21, characterized in that it is capable of forming a depot at the injection site.

23. Process for the preparation of:
drugs, particularly for administration by the oral, nasal, vaginal, ocular, subcutaneous, intravenous, intramuscular, intradermal, intraperitoneal or intracerebral route, it being possible for the active principles of these drugs to be in particular proteins, glycoproteins, proteins bonded to one or more polyalkylene glycol chains, peptides, polysaccharides, liposaccharides, oligonucleotides, polynucleotides and small hydrophobic, hydrophilic or amphiphilic organic molecules;
and/or nutriments;
and/or cosmetic or phytosanitary products,
characterized in that it consists essentially in using at least one polyamino acid according to claim 1.
